(19) United States
(12) Patent Application Publication
(10) Pub. No.: US 2006/0241089 A1
(43) Pub. Date: Oct. 26, 2006

(54) Dipyridamole, acetylsalicylic acid, and angiotensin II antagonist pharmaceutical compositions

(75) Inventors: James C. Gilbert, Bethlehem, CT (US); Lutz Hilbrich, Wiesbaden (DE); Axel Riedel, Maselheim (DE); David Michael Humphreys, Camberley (GB)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 11/478,184

(22) Filed: Jun. 29, 2006

Related U.S. Application Data
(63) Continuation of application No. 10/770,971, filed on Feb. 3, 2004, now abandoned.
(60) Provisional application No. 60/447,090, filed on Feb. 13, 2003.
(60) Provisional application No. 60/503,179, filed on Sep. 16, 2003.

(30) Foreign Application Priority Data
Feb. 13, 2003 (DE) 103 06 179.7
Aug. 8, 2003 (EP) 03 18212.5

(51) Int. Cl. A61K 31/60; A61K 31/519; A61K 31/4184

(52) U.S. Cl. 514/165; 514/262.1; 514/381

(57) ABSTRACT

A pharmaceutical composition comprising a therapeutically effective amount of: (a) dipyridamole or a pharmaceutically acceptable salt thereof; (b) acetylsalicylic acid; and (c) an angiotensin II antagonist, kits containing these three compounds, and methods for preventing stroke or reducing the risk of stroke or secondary stroke in a patient in need thereof by administering an effective amount of these compounds to the patient.

### RELATED APPLICATIONS

This application is a continuation of U.S. Ser. No. 10/770,971, filed Feb. 3, 2004, which claims benefit of U.S. Ser. No. 60/447,090, filed Feb. 13, 2003, and U.S. 60/503,179 and claims priority to German Application No. 103 06 179.7, filed Feb. 13, 2003, and European Application No. EP 03 18212.5, filed Aug. 8, 2003, each of which is hereby incorporated by reference in its entirety.

### FIELD OF THE INVENTION

This invention relates to a method of preventing stroke or reducing the risk of stroke in a patient in need thereof, especially in a patient at risk for a stroke or a secondary stroke, using dipyridamole in combination with acetylsalicylic acid (ASA) and an angiotensin II antagonist, a pharmaceutical composition comprising a combination of dipyridamole, acetylsalicylic acid, and an angiotensin II antagonist, and the use of dipyridamole for the manufacture of a corresponding pharmaceutical composition comprising a combination of dipyridamole, acetylsalicylic acid, and an angiotensin II antagonist.

### BACKGROUND OF THE INVENTION

Dipyridamole {2,6-bis(diethanolamino)-4,8-dipiperidinopyrimido[5,4-d]pyrimidine}, closely related substituted pyrimido-pyrimidines, and their preparation have been described in, e.g., U.S. Pat. No. 3,031,450. Dipyridamole was introduced as a coronary vasodilator in the early 1960s. It is also well-known to have platelet aggregation inhibitor properties due to the inhibition of adenosine uptake. Subsequently, dipyridamole was shown to reduce thrombus formation in a study of arterial circulation of the brain in a rabbit model. These investigations led to its use as an antithrombotic agent; it soon became the therapy of choice for such applications as stroke prevention, maintaining the patency of coronary bypass and valve-replacement, as well as for treatment prior to coronary angioplasty.

Furthermore, the European Stroke Prevention Study 2 (ESPS-2; J. Neurol. Sci. 1996; 143: 1-13; Neurology 1998; 51: 17-19) proved that treatment by dipyridamole alone was as effective as low-dose aspirin in the reduction of stroke risk, and combination therapy with dipyridamole and aspirin was more than twice as effective as aspirin alone.

Dipyridamole appears to inhibit thrombosis through multiple mechanisms. Early studies showed that it inhibits the uptake of adenosine, which was found to be a potent endogenous anti-thrombotic compound. Dipyridamole was also shown to inhibit cyclic AMP phosphodiesterase, thereby increasing intracellular c-AMP.

Dipyridamole also has antioxidant properties (Free Radic. Biol. Med. 1995; 18: 239-247) that may contribute to its antithrombotic effect. When oxidized, low density lipoproteins become recognized by the scavenger receptor on macrophages, which is assumed to be the necessary step in the development of atherosclerosis (Ann. Rev. Med. 1992; 43: 219-25).

The inhibition of free radical formation by dipyridamole has been found to inhibit fibrinogenesis in experimental liver fibrosis (Hepatology 1996; 24: 855-864) and to suppress oxygen radicals and proteinuria in experimental animals with aminonucleoside nephropathy (Eur. J. Clin. Invest. 1998; 28: 877-883; Renal Physiol. 1984; 7: 218-226). Inhibition of lipid peroxidation also has been observed in human nonneoplastic lung tissue (Gen. Pharmacol. 1996; 27: 855-859).

Angiotensin (ANG) II plays a major role in pathophysiology, especially as the most potent blood pressure increasing agent in humans. ANG II antagonists therefore are suitable for treating elevated blood pressure and congestive heart failure in a mammal. Examples of ANG II antagonists are described in EP-A-0 502 314, EP-A-0 253 310, EP-A-0 323 841, EP-A-0 324 377, U.S. Pat. No. 4,355,040, and U.S. Pat. No. 4,880,804. Specific ANG II antagonists are sartans such as candesartan, eprosartan, irbesartan, losartan, telmisartan, or valsartan, furthermore, olmesartan and tasosartan.

It is known that ANG II, besides its blood pressure increasing effect, additionally features growth promoting effects, contributing to left ventricular hypertrophy, vascular thickening, atherosclerosis, renal failure, and stroke. Bradykinin, on the other hand, exerts vasodilating and tissue protective actions, as disclosed, for instance, by W. Wienen et al., Antihypertensive and Renoprotective Effects of Telmisartan After Long Term Treatment in Hypertensive Diabetic (D) Rats, 2nd. Int. Symposium on Angiotensin II Antagonism, Feb. 15-18, 1999, The Queen Elizabeth II Conference Center, London, UK, Book of Abstracts, Abstract No. 50.

Losartan and irbesartan provide a renoprotective effect found within first clinical trials, as disclosed, for instance, by S. Andersen et al., Renoprotective Effects of Angiotensin II Receptor Blockade in Type 1 Diabetic Patients with Diabetic Nephropathy, Kidney Int. 57 (2), 601-606 (2000).

ANG II antagonists selectively block the AT₁, receptor, leaving the AT₂ receptor, which plays a role in anti-growth and tissue regenerative actions, unopposed. Completed clinical trials with ANG II antagonists appear to display similar blood pressure reducing and tissue protective effects as with ACE inhibitors, as disclosed, for instance, by D.H.G. Smith et al., Once-Daily Telmisartan Compared with Enalapril in the Treatment of Hypertension, Adv. Ther. 1998, 15: 229-240, and by B.E. Karlberg et al., Efficacy and Safety of Telmisartan, a Selective ATI Receptor Antagonist, Compared with Enalapril in Elderly Patients with Primary Hypertension, J. Hypertens. 1999, 17: 293-302.

EP-A-1 013 273 discloses the use of AT₁ receptor antagonists or AT₂ receptor modulators for treating diseases associated with an increase of AT₁ receptors in subepithelial area or increase of AT₂ receptors in the epithelia, especially for treatment of several lung diseases.

### SUMMARY OF THE INVENTION

It has now surprisingly been found that dipyridamole, when administered in combination with acetylsalicylic acid and an angiotensin II antagonist, provides a stroke preventing effect superior to conventional medications or treatment regimes, for instance, a combination regime of clopidogrel together with acetylsalicylic acid, especially in a patient at risk for a stroke or a secondary stroke.

ASA inhibits aggregation through direct effects on the platelet, specifically by irreversibly acetylating platelet cyclooxygenase, thus inhibiting the production of thromboxane, which is strongly thrombotic. In high doses, however, aspirin crosses over into endothelial cells (N. Engl. J. Med. 1984; 311: 1206-1211), where it interrupts the production of prostacyclin, a potent natural inhibitor of platelet aggregation and by-product of the “arachidonic cascade” (N. Engl. J. Med. 1979; 300: 1142-1147). These observations led to the concept of low-dose antiplatelet therapy with ASA to maximize inhibition of thromboxane while minimizing the loss of prostacyclin (Lancet 1981; 1: 969-971). In the method of prevention according to the invention, a combination of low-dose ASA with dipyridamole and an angiotensin II antagonist is preferred.

Viewed from one aspect, the present invention provides a method of stroke prevention or reducing the risk of stroke or secondary stroke in a patient in need thereof, especially in a patient with elevated risk for stroke, e.g., in hypertensive patients or patients suffering from cerebrovascular disorders, said method comprising administering to said patient an effective amount of a pharmaceutical composition comprising dipyridamole or a pharmaceutically acceptable salt thereof in combination with ASA and an angiotensin II antagonist. The main risk for a second stroke is a prior stroke due to degenerative processes in the wall of blood vessels supplying the brain. Patients at high risk of a second stroke with all its consequences readily seek preventive treatment. The vascular pathobiology of ischemic stroke is multiple and antithrombotic mechanisms in the cerebrovascular microenvironment beyond platelet inhibition seem to be potentially disease-modifying and a means of reducing ischemic stroke.

Viewed from a second aspect, the present invention provides a pharmaceutical composition comprising dipyridamole or a pharmaceutically acceptable salt thereof in combination with ASA and an angiotensin II antagonist, adapted for simultaneous, separate, or sequential administration.

The pharmaceutical composition according to the invention is meant to comprise a fixed dose combination comprising the active ingredients in one formulation together as well as a kit of parts comprising:
(a) a first container containing a pharmaceutical composition comprising a therapeutically effective amount of dipyridamole;
(b) a second container containing a pharmaceutical composition comprising acetylsalicylic acid and a pharmaceutically acceptable carrier; and
(c) a third container containing a pharmaceutical composition comprising an angiotensin II antagonist.

Viewed from a different aspect, the present invention provides the use of dipyridamole or a pharmaceutically acceptable salt thereof in combination with ASA and an angiotensin II antagonist for the manufacture of a pharmaceutical composition for stroke prevention or reducing the risk of stroke or secondary stroke in a patient in need thereof.

### DETAILED DESCRIPTION OF THE INVENTION

The invention provides a new and improved approach for stroke prevention or reducing the risk of stroke or secondary stroke in a patient in need thereof, especially in a patient with elevated risk for stroke, comprising administering to the patient an effective amount of a pharmaceutical composition containing as active ingredients dipyridamole or a pharmaceutically acceptable salt thereof in combination with ASA and an angiotensin II antagonist.

In the method of the invention, any of the oral dipyridamole retard (extended release), instant, or the parenteral formulations on the market may be used, the retard formulations being preferred, for instance, those available under the trademark PERSANTIN®, or, already in combination with ASA, the formulations available under the trademark ASASANTIN® or AGGRENOX®. Suitable dipyridamole retard formulations are disclosed in EP-A-0032562, instant formulations are disclosed in EP-A-0068191, and combinations of ASA with dipyridamole are disclosed in EP-A-0257344, which are herein incorporated by reference. Any angiotensin II antagonist known in the art may be used in the method of prevention of the invention, e.g., the sartans such as candesartan, eprosartan, irbesartan, losartan, telmisartan (sold under the trademark MICARDIS®), valsartan, olmesartan, or tasosartan, including the salts thereof or polymorphs thereof, using for instance the dosages disclosed in ROTE LISTE® 2003, Editio Cantor Verlag Aulendorf, Germany, or in Physician's Desk Reference, 57 edition, 2003.

In the method of prevention according to the invention, a plasma level of dipyridamole of about 0.2 μmol/L to 5 μmol/L, preferably of about 0.5 μmol/L to 2 μmol/L or particularly of about 0.8 μmol/L to 1.5 μmol/L may be maintained. Dipyridamole can be administered orally in a daily dosage of 50 mg to 750 mg, preferably 100 mg to 500 mg, and most preferred 200 mg to 450 mg, for instance, 200 mg twice a day.

With respect to ASA, this component of the ternary medication may be administered orally in a daily dosage of 10 mg to 200 mg, preferably 25 mg to 100 mg, and most preferred 30 mg to 75 mg, for instance, 25 mg twice a day.

With respect to the third component, the angiotensin II antagonist, telmisartan is preferred. This component can be administered orally in a daily dosage of 10 mg to 200 mg, for instance, in a daily dosage of 20 mg, 40 mg, 80 mg, or 160 mg, preferably in a daily dosage of 40 mg to 160 mg, and most preferred 60 mg to 100 mg, for instance, 20 mg or 40 mg once a day.

A specific method of prevention according to the invention comprises the combination of dipyridamole administered orally 200 mg twice a day, ASA administered orally 25 mg twice a day, and telmisartan administered orally 20 mg, 40 mg, or 80 mg once a day.

With respect to all aspects of the invention the combination of dipyridamole, ASA, and telmisartan is preferred, especially in the oral dosages indicated hereinbefore as most preferred.

All of the patents, patent applications, or other references referred to herein are hereby incorporated by reference in their entireties.

We claim:

1. A pharmaceutical composition comprising a therapeutically effective amount of:
(a) dipyridamole or a pharmaceutically acceptable salt thereof;
(b) acetylsalicylic acid; and
(c) an angiotensin II antagonist.

2. The pharmaceutical composition according to claim 1, wherein the angiotensin II antagonist is candesartan, eprosartan, irbesartan, losartan, telmisartan, valsartan, olmesartan, or tasosartan, or a salt or polymorph thereof.

3. The pharmaceutical composition according to claim 1, wherein the angiotensin II antagonist is telmisartan, or a salt or polymorph thereof.

4. The pharmaceutical composition according to claim 1, further comprising a pharmaceutically acceptable excipient or carrier.

5. The pharmaceutical composition according to claim 2, further comprising a pharmaceutically acceptable excipient or carrier.

6. The pharmaceutical composition according to claim 3, further comprising a pharmaceutically acceptable excipient or carrier.

7. A kit comprising:
(a) a first container containing a first pharmaceutical composition comprising dipyridamole or a pharmaceutically acceptable salt thereof;
(b) a second container containing a second pharmaceutical composition comprising acetylsalicylic acid; and
(c) a third container containing a third pharmaceutical composition comprising an angiotensin II antagonist,
wherein the total amount of (a), (b), and (c) is a therapeutically effective amount.

8. The kit according to claim 7, wherein the angiotensin II antagonist is candesartan, eprosartan, irbesartan, losartan, telmisartan, valsartan, olmesartan, or tasosartan, or a salt or polymorph thereof.

9. The kit according to claim 7, wherein the angiotensin II antagonist is telmisartan, or a salt or polymorph thereof.

10. The kit according to claim 7, wherein the first pharmaceutical composition further comprises a pharmaceutically acceptable excipient or carrier.

11. The kit according to claim 7, wherein the second pharmaceutical composition further comprises a pharmaceutically acceptable excipient or carrier.

12. The kit according to claim 7, wherein the third pharmaceutical composition further comprises a pharmaceutically acceptable excipient or carrier.

13. The kit according to claim 7, wherein each of the first, second, and third pharmaceutical compositions further comprise a pharmaceutically acceptable excipient or carrier.

14. The kit according to claim 7, further comprising instructions for simultaneous, separate, or sequential administration of the first, second, and third pharmaceutical compositions.

15. A kit comprising:
(a) a first container containing a first pharmaceutical composition comprising dipyridamole or a pharmaceutically acceptable salt thereof and acetylsalicylic acid; and
(b) a second container containing a second pharmaceutical composition comprising an angiotensin II antagonist,
wherein the total amount of (a) and (b) is a therapeutically effective amount.

16. The kit according to claim 15, wherein the angiotensin II antagonist is candesartan, eprosartan, irbesartan, losartan, telmisartan, valsartan, olmesartan, or tasosartan, or a salt or polymorph thereof.

17. The kit according to claim 15, wherein the angiotensin II antagonist is telmisartan, or a salt or polymorph thereof.

18. The kit according to claim 15, wherein the first pharmaceutical composition further comprises a pharmaceutically acceptable excipient or carrier.

19. The kit according to claim 15, wherein the second pharmaceutical composition further comprises a pharmaceutically acceptable excipient or carrier.

20. A method for preventing stroke or reducing the risk of stroke or secondary stroke in a patient in need thereof, comprising administering to the patient an effective amount of dipyridamole or a pharmaceutically acceptable salt thereof, acetylsalicylic acid, and an angiotensin II antagonist.

21. The method according to claim 20, wherein the angiotensin II antagonist is candesartan, eprosartan, irbesartan, losartan, telmisartan, valsartan, olmesartan, or tasosartan, or a salt or polymorph thereof.

22. The method according to claim 20, wherein the angiotensin II antagonist is telmisartan, or a salt or polymorph thereof.
